(19) United States
(12) Patent Application Publication
(10) Pub. No.: US 2006/0272392 A1
(43) Pub. Date: Dec. 7, 2006

(54) Relative humidity probe for concrete

(75) Inventor: Howard M. Kanare, Wilmette, IL (US)

Correspondence Address: FOLEY & LARDNER LLP, 321 NORTH CLARK STREET, SUITE 2800, CHICAGO, IL, 60610-4764, US

(73) Assignee: Construction Technology Laboratories, Inc.

(21) Appl. No.: 11/441,876

(22) Filed: May 26, 2006

Related U.S. Application Data
(63) Continuation-in-part of application No. 11/001,729, filed on Dec. 2, 2004.

(51) Int. Cl. G01N 33/38

(52) U.S. Cl. 73/29.02; 73/73

(57) ABSTRACT

A probe for determining the relative humidity and temperature of a substance such as a concrete floor. The probe is adapted to be inserted into a hole in the substance being tested. The probe has a head portion and a tail portion. A display is provided on the head portion to provide a user with a visual indication of the relative humidity and temperature within the substance. The entire probe structure is designed to be contained within the hole without the need for any components protruding out of the hole to cause an obstruction.

### RELATED APPLICATIONS

The present application is a continuation-in-part of U.S. patent application Ser. No. 11/001,729 filed Dec. 2, 2004 and which is hereby incorporated by reference.

### FIELD OF THE INVENTION

The present invention relates generally to the field of humidity probes, and more particularly to relative humidity probes for concrete.

### BACKGROUND OF THE INVENTION

Concrete is a common substrate for many commercial, industrial, and residential floors. Many types of floor coverings including wood, carpet, vinyl tile, sheet vinyl, and linoleum are commonly placed on concrete floors. Coatings such as epoxies, polyurethanes, and polymer-terrazzo, are widely used in commercial, governmental, educational, manufacturing, and health care facilities. These types of floor coverings are sensitive to moisture and prone to failure when excessive moisture is present. In addition to the floor covering materials, many modern water-based adhesives are prone to failure when excessive moisture and high pH are present. Furthermore, moisture also promotes fungal growths that can create significant odor and health problems. Typical moisture-related failures of floor coverings are curling, cupping, doming, shrinkage, blistering, and adhesion loss, all potentially leading to trip-and-fall hazards. In retail establishments, adhesive oozing at tile seams is unsightly and attracts dirt, leading to cleaning problems. In previous decades, the most commonly used asphaltic cut-back adhesives for vinyl tile were relatively insensitive to moisture. Polymer coatings and polymer terrazzo fail via osmotic blistering. Excessive moisture in concrete floors often delays construction, the installation of finish flooring and furniture, and ultimately occupancy, creating major problems for the floor covering and coating industries.

To avoid moisture problems, floor covering and coating manufacturers specify a maximum moisture vapor emission rate (MVER) for concrete floors on which their products will be installed. The industry standard MVER specification is 3 to 5 lbs./1,000 sq. ft./24 hrs. when measured by ASTM F1869, Standard Test Method for Measuring Moisture Vapor Emission Rate of Concrete Subfloor Using Anhydrous Calcium Chloride. This test is widely used: manufacturers of test kits have indicated that more than 300,000 test kits are sold annually in the United States. However, this test method has many interferences and shortcomings. Recognizing these problems, ASTM Committee F-6 on Resilient Floor Coverings adopted ASTM F2170-02, Standard Test Method for Determining Relative Humidity in Concrete Floor Slabs Using in situ Probes. This test method employs an electronic humidity sensor placed into a drilled hole in concrete, connected by a cable to a hand-held meter. This method was drawn from recent experience in Scandinavia and England, where relative humidity measurements in floors have been used for two decades. Building codes and codes of practice for installation of flooring in Europe have standards based on relative humidity. Flooring manufacturers in the United States are aware of this test method and are beginning to publish requirements for moisture as measured by relative humidity probes.

Moisture testing using relative humidity probes has been well established and used for over 50 years in the United States and Europe. Two types of methods are used. In one, exemplified by ASTM F2420, a rigid insulated box called a “hood” is secured to a concrete floor slab using sealant compound to create a hermetically sealed chamber into which a moisture sensing probe is inserted. In the other method, exemplified by ASTM F2170, a hole is drilled into a concrete slab and a moisture sensing probe is inserted into the hole. Both methods require the sensing probes to remain in place a minimum of 72 hours to allow moisture within the test volume to equilibrate with moisture in the concrete, thereby producing an accurate reflection of the percent relative humidity in that volume of concrete immediately adjacent to the sensing element. The requirement for the 72 hour equilibration period is based on work at two internationally known research centers: the Swedish Center for Building Research (Molina 1990) and Lund University (Hedenblad, 1997).

This relatively long equilibration period creates several problems for users. The user must travel to a jobsite, prepare and install the moisture sensing apparatus, leave the jobsite, and return three days later to record the test results. There is expense and time associated with each test that could be avoided or minimized if the test results could be obtained within a short time after installing the moisture sensor probes.

Commercially available relative humidity probes suffer from several drawbacks. First, they require time to equilibrate with the surrounding concrete. ASTM F2170 currently requires that drilled holes sit undisturbed for 72 hours before measurement. The larger the diameter of hole drilled, the longer the time necessary for the hole to equilibrate. It is currently believed that available probes require this long waiting period due to at least three factors. First, the heat generated by drilling disturbs the moisture equilibrium in the region of the drilled hole. A 1990 study by the Swedish Cement and Concrete Research Institute, a leader in the field, found that avoiding heat generation while drilling would be desirable. See, Molina, Larissa, “Measurement of high humidity in cementitious material at an early age”, Swedish Cement and Concrete Research Institute CBI Report, 3:30, pg. 59, 64. Time is required for the hole to re-equilibrate at service temperature and for moisture to equilibrate by diffusion within the region of the hole. Second, the “dead volume” within these probes, due to their size, requires time for moisture to diffuse and equilibrate within the probes. Third, temperature differences between the probe and the concrete require time for equilibration due to the heat capacity of the probes. All of these factors require the testing agency to wait several days to obtain test results, often when construction schedules are tight. Many of these problems stem from the relatively large size of current probes being used.

A second problem is that the current generation of instruments meeting ASTM F2170 have RH probes that are placed in concrete and separate hand held meters to which they are connected to obtain results. The meters are bulky, expensive, and can only be used with one RH probe at a time. One brand of meter requires that calibration factors for each probe be manually entered into the hand held meter one-at-a-time before use, and only up to ten such factors can be accommodated in the meter. A user must be familiar with the handheld meter to be able to determine the relative humidity.

A third problem is that the current generation of probes all protrude from the drilled holes in the concrete, making them susceptible to damage from construction traffic or building usage, or even present safety dangers to construction workers or other passersby. Probes often are broken or damaged beyond repair due to this problem. In addition, this protrusion makes current probes unsuitable for long term use or for intermittent use throughout the life of a building.

A fourth problem is that the probes require cylindrical sleeves be inserted into the holes to shield the probes from concrete except at the bottom of the hole where it is desired to make the RH measurement. Sleeves must be purchased separately and carried to the jobsite for installation. Sleeves wear out after several uses and must be replaced.

Thus, there is a need for a probe able to make accurate relative humidity measurements following the procedures outlined in ASTM F2170, but make them much more rapidly and conveniently, with less likelihood of damage to the RH measuring devices.

It is to be understood that both the foregoing general description and the following detailed description are exemplary and explanatory only, and are not restrictive of the invention as claimed.

### SUMMARY OF THE INVENTION

The present invention solves the several problems of prior art probes enumerated above. In one embodiment, the present invention relates to a humidity probe having a housing with a head portion and a tail portion. An electronics module is disposed within the housing. Associated with the head portion and in communication with the electronics module is a display. A relative humidity sensor is disposed within the tail portion of the housing and in operative communication with an exterior environment and the electronics module.

In another exemplary embodiment, the present invention relates to a method of determining properties of a substance, such as concrete. The method includes the step of boring a hole having a bottom and a top in the substance. A probe having a display is inserted into the hole. The probe is positioned so that it is substantially disposed within the hole with the display located at the top of the hole. A relative humidity of the substance at the bottom of the hole is determined and displayed on the display.

In another exemplary embodiment, the present invention relates to a method of equilibrating a hole in a sample and determining the relative humidity of the sample. A hole is bored in the sample and a vacuum device attached. The vacuum results in excess moisture evaporating, allowing the hole to equilibrate quicker. A humidity probe is then positioned within the hole, below the top of the surface. A sample area is sealed off from the environment outside the hole and the relative humidity is determined.

In another exemplary embodiment, the present invention relates to a kit for boring a hole in a substance and determining relative humidity therein. The kit includes a drill bit having a lead portion and a reamer portion. The kit further includes a probe. The probe has a housing having an elongated portion and a head portion. An electronics module is disposed within the housing. A display, which is in communication with the electronics module, is located in the head portion. A relative humidity sensor is disposed within the elongated portion of the housing and in operative communication with an exterior environment and the electronics module. The drill bit is adapted to drill a lead hole and counterbore corresponding to the elongated portion and head portion respectively wherein the probe is countersunk within the hole.

These and other features, aspects, and advantages of the present invention will become apparent from the following description, appended claims, and the accompanying exemplary embodiments shown in the drawings, which are briefly described below.

### BRIEF DESCRIPTION OF THE DRAWINGS

FIG. 1 illustrates a cross-sectional view of one embodiment of a probe in accordance with the principles of the present invention;

FIG. 2 illustrates a top view of the probe of FIG. 1;

FIG. 3 illustrates a bottom view of the probe of FIG. 1;

FIG. 4 illustrates a cross sectional view of a probe of the present invention, depicting a first half;

FIG. 5 illustrates a cross sectional view of the probe of the FIG. 4, depicting the remaining half;

FIG. 6 illustrates a top perspective view of the probe of FIGS. 4 and 5;

FIG. 7 illustrates an organizational chart of the electronics of the present invention;

FIG. 8 illustrates a drill bit in accordance with the principles of the present invention;

FIG. 9 illustrates a brush attachment for use with the present invention;

FIG. 10 is a graph depicting the time to equilibrate a device in accordance with the principles of the present invention;

FIG. 11 is a cross-sectional view of one embodiment of the present invention in a hole, where the ridges isolate the bottom portion of the hole;

FIG. 12 is a graph depicting the equilibration time for four types of probes in a 95% relative humidity concrete sample;

FIG. 13 is a graph depicting the same four types of probes of FIG. 12 in a 75% relative humidity concrete sample after the application of a vacuum for 2 minutes;

FIG. 14 is a graph illustrating the impact of various vacuum lengths on equilibration time in high relative humidity concrete of a probe of one embodiment of the present invention; and

FIG. 15 is a graph illustrating the impact of various vacuum lengths on equilibration time in low relative humidity concrete of a probe of one embodiment of the present invention.

### DETAILED DESCRIPTION OF THE PREFERRED EMBODIMENTS

The present invention relates to a relative humidity probe having a small form factor for placement within a sampling hole in a material, thus being less susceptible to damage while in use than prior art sensors. The present invention comprises a relative humidity sensor with an integrated cylindrical sleeve and an integrated output display, all in one object to allow the device to rest below the surface of the substance being tested, such as below the surface of a concrete floor.

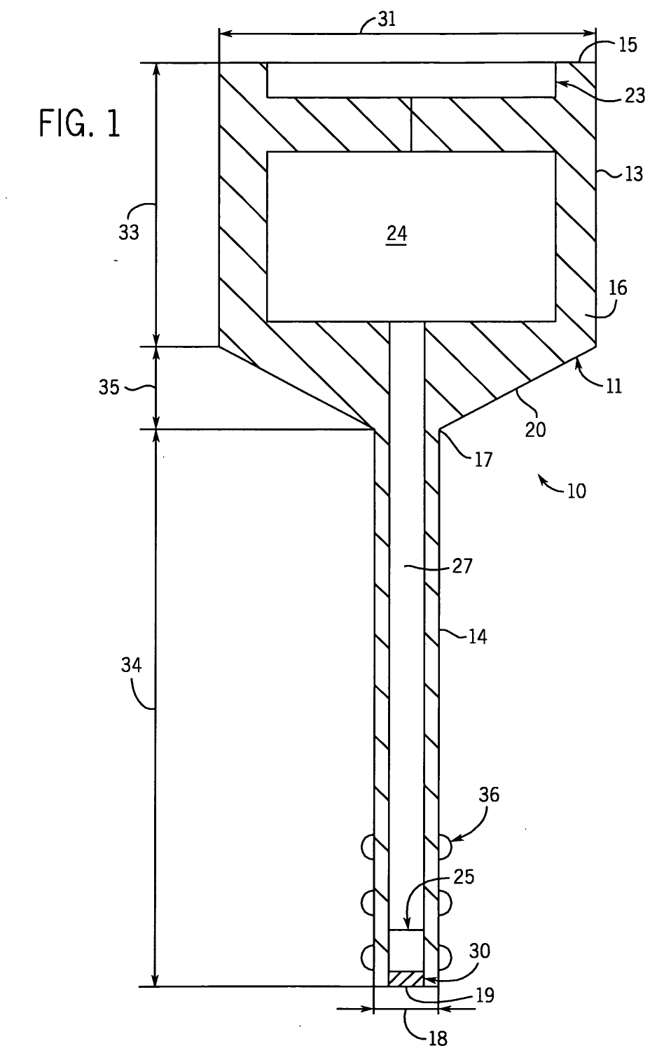

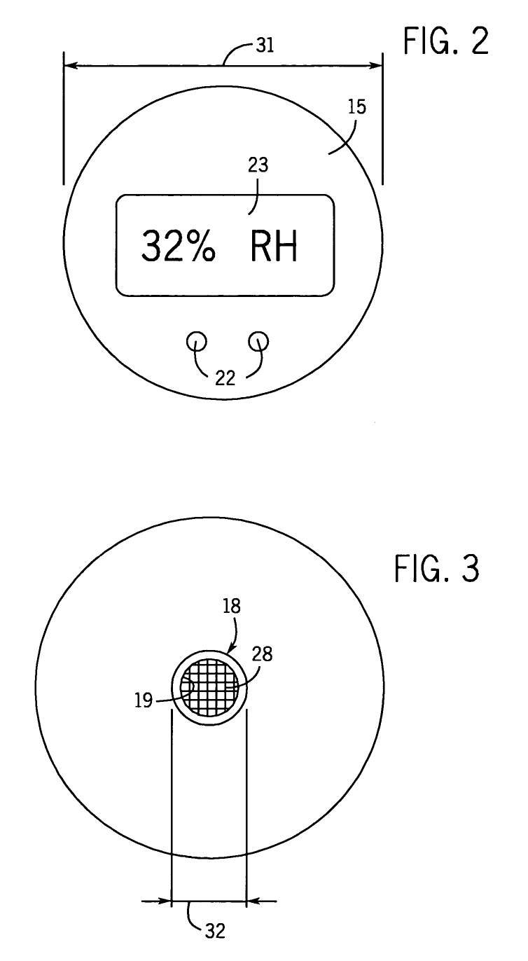

Referring to the figures, one embodiment of a relative humidity sensor probe in accordance with the principles of the present invention is illustrated in FIGS. 1-3. As shown in FIG. 1, the probe 10 comprises a housing 11 forming an outer shell within which the components of the probe 10 are disposed. The housing 11, itself, has a head portion 13 and an elongated or tail portion 14. The head portion 13 has a top end 15 and connecting end 16. The top end 15 is, when the probe 10 is in use, facing out of the hole so as to be visible to a user. The connecting end 16 is connected to the tail portion 14. The tail portion 14 has a detection end 18 and a connecting end 17 which is connected to the connecting end 16 of the head portion 13. The detection end 18 of the tail portion 14 includes an aperture 19 through which a relative humidity sensor 25 (discussed below) is in communication with the environment outside of the probe 10.

In one preferred embodiment, the housing 11 has a circular cross section. In an exemplary embodiment, the head portion 13 has a diameter different from that of the tail portion 14. Preferably, the head portion 13 has a diameter greater than the tail portion 14. In one exemplary embodiment, the head portion 13 has a diameter substantially larger than the diameter of the tail portion 14. In one embodiment, the head portion 13 has a diameter of about 20 millimeters and the tail portion 14 has a diameter of about 4 millimeters. Thus, in one embodiment, the probe 10 has the tail portion 14 (i.e., probe tip) that is approximately one-fourth the diameter of existing probes, thus requiring a drilled hole that is only one-fourth the surface area and one-sixteenth the volume required by current instruments. Thus, drilled holes of the present invention are smaller in diameter and equilibrate rapidly, a feature much desired for concrete moisture testing.

In one embodiment, the head portion 13 and the tail portion 14 are connected via a tapered region 20. The tapered region transitions from having a perimeter equal to the perimeter of the connecting end 16 of the head portion 13 to a perimeter equal to the perimeter of the detection end 18 of the tail portion 14.

In an exemplary preferred embodiment, the head portion 13 has a height which is less than the height of the tail portion 14. In one embodiment, the head portion 13 has a height which is less than about half of the height of the tail portion 14. In one embodiment, the taper region 20 has a height less than that of the head portion 13. In one embodiment, the head portion 13 has a height of about 15 millimeters, the tail portion 14 has a height of about 30 millimeters, and the tapered region 20 has a height of about 5 millimeters.

The head portion 13 contains a display 23. In one exemplary embodiment, the display 23 is disposed within the head portion 13 of the housing 11. In another exemplary embodiment, the display 23 is disposed on top of the head portion 13. The display 23 is adapted to provide a visual indication of the relative humidity determined by the probe 10. In an exemplary embodiment, the display 23 is further adapted to provide visual indication of other information such as temperature (in degrees Celsius or degrees Fahrenheit), time, date, location identification number, probe identification number, etc. In one preferred embodiment, the display 23 is a liquid crystal display (LCD). In another embodiment, the display 23 is a Light Emitting Diode (LED) type display. Various other types of display technologies can be used in accordance with the principles of the present invention without departing from the scope of the invention.

The display 23 is in communication with an electronics module 24. FIG. 7 illustrates the components in electrical communication with the electronics module 24. The electronics module 24 operates as a processor, translating signals from a relative humidity sensor 25 into a signal to the display 23. In one embodiment, the electronics module 24 includes a power source such as a battery 26.

The relative humidity sensor 25 is disposed within the tail portion 14. In one embodiment, the relative humidity sensor 25 is located substantially at the detection end 18 of the tail portion 14 so as to be in operative communication with the environment outside of the probe 10. In one embodiment, best shown in FIG. 2, the aperture 19 is covered by a perforated screen 28. In an exemplary embodiment, a vapor permeable member 30 is provided between the relative humidity sensor 25 and the perforated screen 28 through which the relative humidity sensor 25 is able to detect the water vapor content of the environment outside of the probe 10. In one embodiment, unlike conventional probes, the relative humidity sensor 25 is placed in direct contact with the outside environment through a permeable or semi-permeable membrane change to a permeable or semi-permeable membrane such as vapor permeable member 30, with no housing or sleeve to create additional dead space. The positioning of the relative humidity sensor 25 and the use of ridges 49, as described below, result in less dead space than conventional probes, which results in reduced equilibration time. Most conventional probes are designed to require an outer sleeve into which the housing containing the sensor is placed, this results in additional time to equilibrate due to the dead volume between the sensor and the sample.

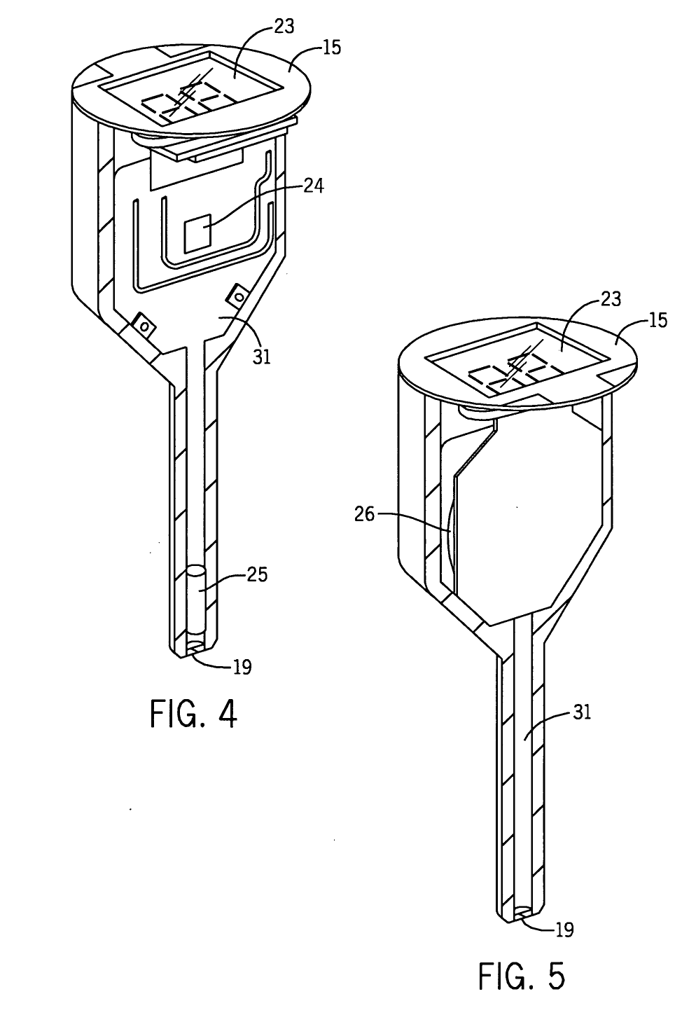

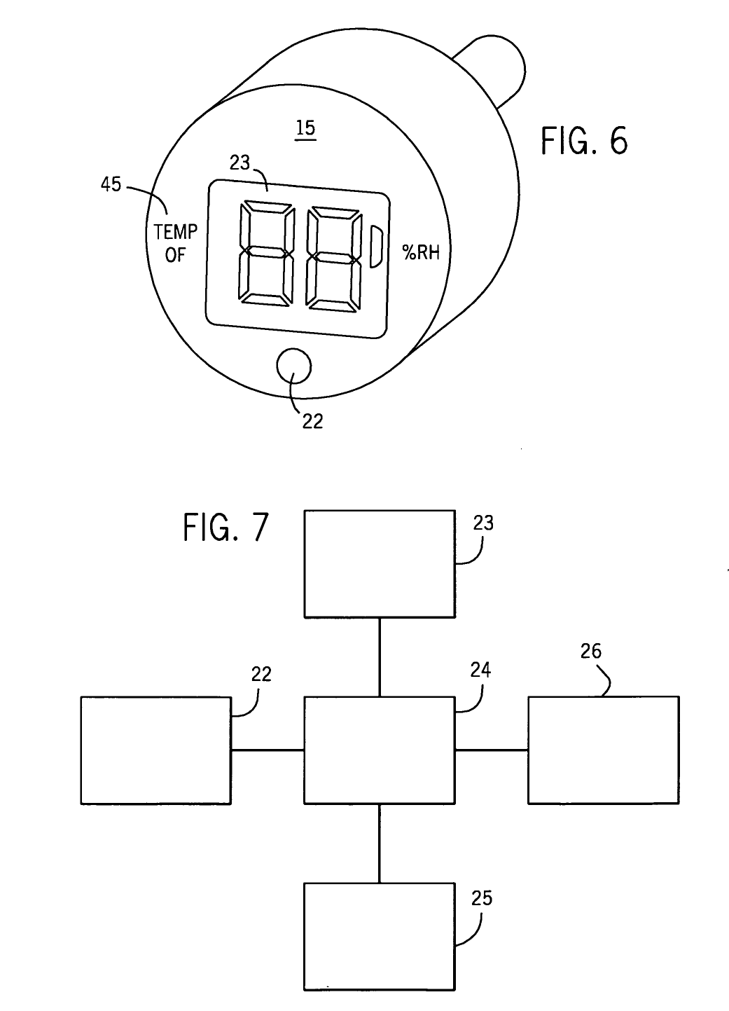

In one embodiment shown in FIG. 1, a hollow core 27 is provided within the housing 11. The hollow core 27 runs from the electronics module 24 in the head portion 13 through the tail portion 14 to the detection end 18 forming the aperture 19 therein. In one exemplary embodiment depicted in FIGS. 4-6, a printed circuit board 31 connects the relative humidity sensor 25 with the electronics module 24. In an exemplary embodiment, the printed circuit board 31 is disposed within the hollow core 27. In one embodiment, a battery 26 is provided on one side of the circuit board 31 printed in electrical communication with the electronics module 24, relative humidity sensor 25, and display device 23 via the printed circuit board 31.

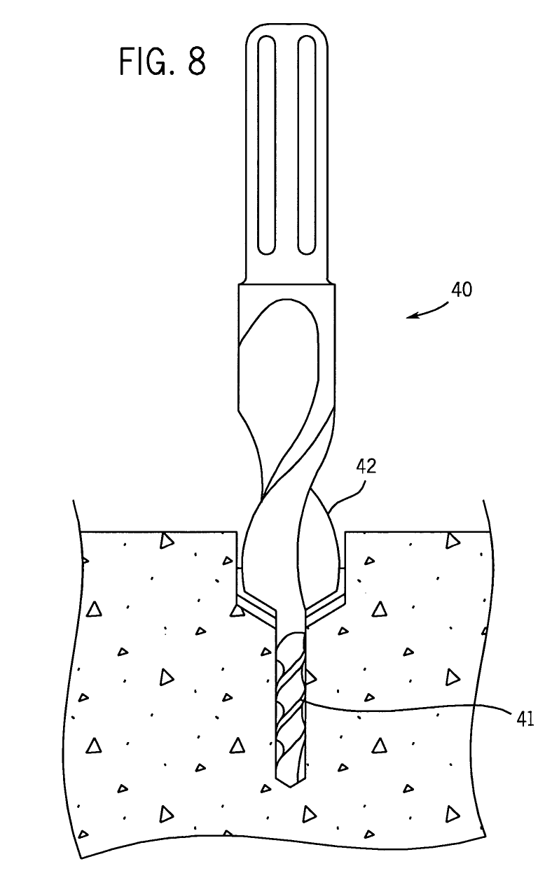

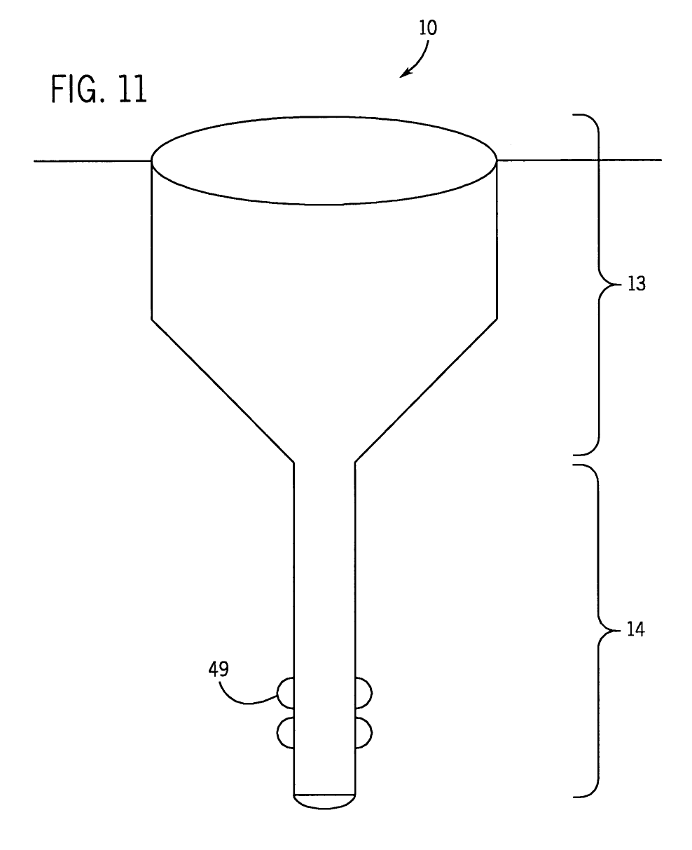

One embodiment, shown in FIG. 11, relates to systems and methods for isolating a sample space with a hole in a surface being tested. In an exemplary embodiment, the housing 11 includes at least one ridge or fin 49. The ridge 49 circumscribes the tail portion 14 substantially near the bottom of the tail portion (i.e., near the humidity sensor 25). The ridge 49 is designed to sealingly engage the side of the hole to seal the bottom portion of the hole from the remainder of the hole and the environment outside the hole. The area sealed off by the ridge 49 forms a test area which will equilibrate more quickly and accurately with the surrounding surface material. The lack of dead space within the tail portion 14 of the probe 10, due to the placement of the relative humidity sensor 25 substantially at the end of the probe 10 exposed to the outside of the probe 10, and the formation of a small test area by the ridges 49 results in a small volume of air that requires equilibration in comparison to conventional systems. As shown in FIG. 8, a further aspect of the system of the present invention is directed to a drill bit 40 for boring out a pilot hole and a counterbore into which the probe 10 may be inserted. The drill bit 40 is designed to drill both the pilot hole and counterbore hole at once. The drill bit 40 has a leading tip 41 and a reamer portion 42. The shape and size of the drill bit 40 corresponds to the shape and size of the probe 10.

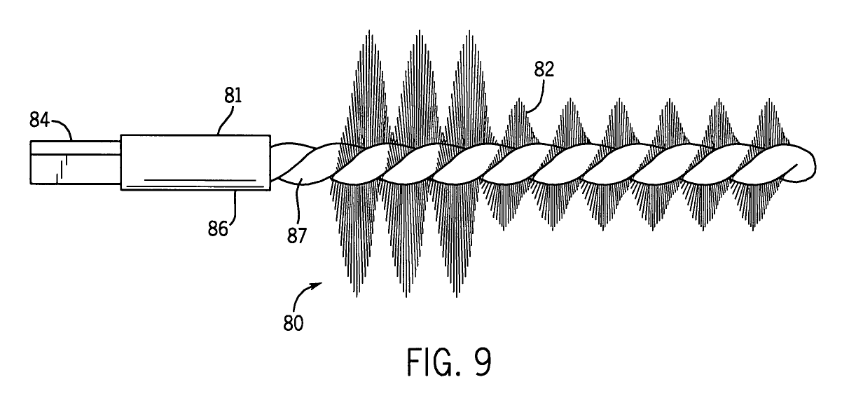

A further aspect of the invention relates to a cleaner attachment that permits rapid and thorough cleaning of the drilled hole before inserting the probe 10. As shown in FIG. 9, the cleaner attachment 80 includes a shaft 81 and a brush portion 82. The shaft 81 includes a base portion 84 for engagement with a handle and an upper portion 86 for supporting the brush portion 82. In one embodiment, the upper portion 86 comprises two helically wound strands 87, such as metal wire, of sufficient thickness to provide rigid support. The brush portion 82 comprises strands such as wire, disposed perpendicular to and between the helically wound strands 87. In one embodiment, the brush portion 82 is tapered such as to provide a conical shape when the cleaner attachment 80 is rotated. In use, the cleaner attachment 80 is placed into a drill, inserted in the hole drilled for the probe 10, and is rotated to remove any debris in the hole. The removal of the debris increases the accuracy of the probe's results and can aid in reducing the time required for the hole to reach equilibrium.

In one embodiment, a method measures the relative humidity of a solid material wherein a vacuum is used to equilibrate a sample hole in the solid material prior to testing. It had been assumed that frictional heating during drilling upset moisture equilibrium in the concrete and caused the long time to equilibration. This embodiment involves applying at least a partial vacuum to the volume of the drilled hole. Then the excess moisture is allowed to evaporate under the reduced pressure, therefore evaporating more quickly than if no vacuum was applied, leading to subsequent rapid equilibration. In accordance with the principles of the present invention, high-moisture slabs can achieve equilibrium from several minutes to less than an hour which represents a very significant improvement over the existing methods that require at least several days equilibration time. Example 2 further provides discussion of an example of this aspect of the invention.

In one embodiment, the present invention relates to a kit including the probe 10, the drill bit 40, and the cleaner attachment 80. These three devices (drill bit 40, cleaner attachment 80, and probe 10) used in succession, permit a user to quickly drill and clean a hole, insert the probe 10, and obtain accurate relative humidity measurements with only a brief waiting period.

One of ordinary skill in the art will appreciate that the present invention may be used with numerous materials. In a preferred embodiment, the present invention is used for detecting moisture in concrete floor slabs. However, the present invention is suitable for measuring moisture in a variety of construction materials, systems, and structures including, but not limited to:
Concrete building elements such as beams, columns, roof decks, and walls
Pavements, runways, concrete flatwork
Building materials such as exterior insulation and finish systems (EIFS), wall cavities, wood structural elements, gypsum concretes, masonry
Use in British Standard BS8203 and ASTM F2420 “Hood” Methods

In one embodiment, the probe 10 can be left embedded in the concrete building element, such as a floor, and reactivated later in the life to measure the moisture condition, for example, if moisture problems appear, or simply to monitor the moisture situation in the building. The probe 10 is designed to be placed below the surface of the material being tested, such as in a bored-out hole. Since it lies totally below the floor surface, or embedded in another element of the building, it is unobtrusive, and not likely to be damaged by traffic or building usage while installed. In an exemplary embodiment, the probe 10 operates in a “standby mode” wherein the device is inactive. A user may activate the probe 10 such as by use of a button on the top of the probe 10. Upon activation, the probe 10 detects the relative humidity. In one embodiment, a button or mechanism is provided to cycle through the various pieces of information which the probe 10 can display (as previously discussed above). In another embodiment, the display 23 automatically cycles through the various pieces of information pausing for a predetermined time to allow a user to observe the information. In one embodiment, the probe 10 includes a mechanism for automatically deactivating the probe 10 after a predetermined period of time.

In an exemplary preferred embodiment, the present invention contains a mechanism to conserve battery life. In one embodiment, the electronics module 24 includes a mechanism which turns the probe 10 off automatically after a certain time period. A user may turn the probe 10 on by actuating a button the top of the head portion 13 of the housing 11. In one embodiment, the probe 10 can be connected via a wireless system to a remote location such as by a RF signal. In one embodiment, the probe is adapted to be in communication with a wireless handheld device.

A non-limiting example is provided below to further illustrate aspects and advantages of the present example is illustrative only and not intended to limit the scope of the invention.

### EXAMPLES

### Example 1

### Equilibration Using One Embodiment of a Probe in Accordance With the Principles of the Present Invention

A 100-mm thick concrete slab was constructed using a concrete mix typical of commercially-available concrete mixes used for floor slab construction. A hole was drilled in the concrete floor slab using the drill bit 40. The hole was brushed and vacuumed to remove dust, and a probe 10 was inserted. Relative humidity and temperature readings were recorded at intervals by visually observing the display 25 from the probe 10.

Table 1 lists of this experiment, indicating the relative humidity and temperature for sampled times after insertion.

TABLE 1
Experimental Results

| Time After | | |
|---|---|---|
| Insertion | Relative Humidity | Temperature |
| (Minutes) | (Percentage) | (° F.) |
| 5 | 82 | 67 |
| 8 | 83 | 67 |
| 10 | 84 | 67 |
| 12 | 84 | 67 |
| 14 | 84 | 67 |
| 16 | 85 | 67 |
| 18 | 85 | 67 |
| 20 | 85 | 67 |
| 24 | 85 | 67 |
| 27 | 85 | 67 |
| 30 | 85 | 67 |
| 35 | 85 | 67 |
| 40 | 85 | 67 |
| 45 | 85 | 67 |

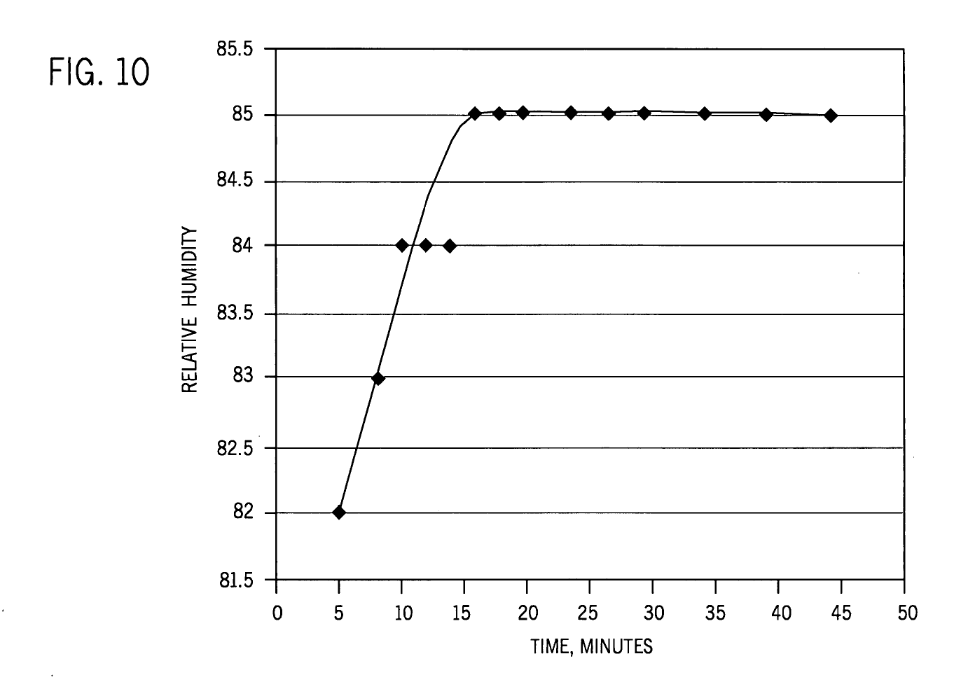

These results are graphed in FIG. 10, as percentage humidity (y-axis) versus time (x-axis). As can be seen both from Table 1 and FIG. 10, the humidity readings equilibrated after 16 minutes and remained so for the remainder of the testing period (45 minutes).

### Example 2

### Comparison of Relative Humidity Equilibration with no Vacuum

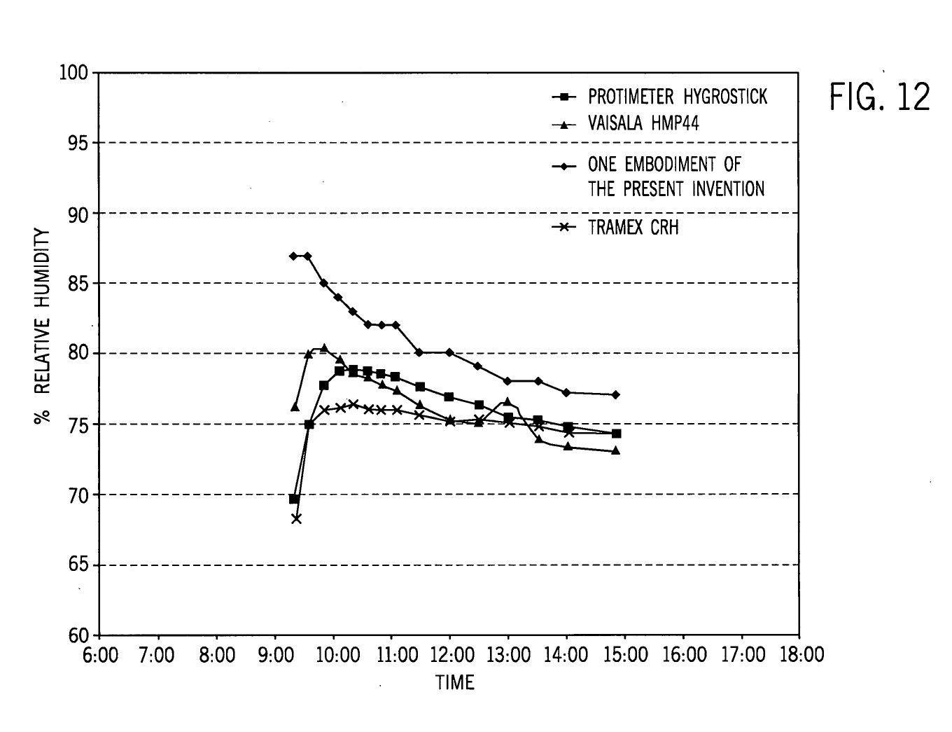

Conventional thought has indicated that holes drilled in concrete or other such surfaces require a period of time to equilibrate with the rest of the surface due to increased local temperature in and surrounding the hole from the frictional forces. A probe of one embodiment of the present invention was utilized (diamond line) along with a Protimeter Hygrostic (square line), a Vaisala HMP44 (triangle line), and a Tramex CRH (x line) commercial humidity probes by being placed in 4 inch thick holes in standard concrete. FIG. 12 illustrates the equibration time in a 95% relative humidity sample, Table 2 containing the data used in generating FIG. 12.

TABLE 2
95% RH no vac, 4 Probes

| | | | | | | | Present |
|---|---|---|---|---|---|---|---|
| | Protimeter | | Vaisala | | | | Invention |
| TIME | RH % | TIME | RH % | TIME | Tramex RH % | TIME | RH % |
| 6:30 | 68 | 6:43 | 77 | 6:38 | 80 | 6:26 | 85 |
| 6:40 | 74.7 | 6:40 | 81.7 | 6:49 | 85.6 | 6:32 | 88 |
| 6:50 | 77.1 | 6:49 | 85.6 | 7:00 | 89 | 6:38 | 88 |
| 7:00 | 80 | 7:00 | 88.6 | 7:15 | 90.6 | 6:49 | 89 |
| 7:15 | 80.7 | 7:15 | 90.4 | 7:30 | 91.5 | 7:00 | 90 |
| 7:30 | 81.9 | 7:30 | 91.3 | 7:45 | 92.1 | 7:15 | 90 |
| 7:45 | 82.9 | 7:45 | 92.2 | 8:00 | 92.5 | 7:30 | 90 |
| 8:00 | 83.6 | 8:00 | 92.5 | 8:15 | 93 | 7:45 | 91 |
| 8:15 | 84.4 | 8:15 | 92.9 | 8:30 | 93 | 8:00 | 91 |
| 8:30 | 84.6 | 8:30 | 92.8 | 8:45 | 93.7 | 8:15 | 92 |
| 8:45 | 85.3 | 8:45 | 93.3 | 9:00 | 94.1 | 8:30 | 92 |
| 9:00 | 85.8 | 9:00 | 93.6 | 9:30 | 94.7 | 8:45 | 93 |
| 9:30 | 86.3 | 9:30 | 94.1 | 10:00 | 94.8 | 9:00 | 93 |
| 10:00 | 86.9 | 10:00 | 94.4 | 10:30 | 95 | 9:30 | 94 |
| 10:30 | 87.1 | 10:30 | 94.7 | 11:00 | 95.4 | 10:00 | 94 |
| 11:00 | 87.5 | 11:00 | 94.7 | 11:30 | 95.7 | 10:30 | 94 |
| 11:30 | 87.8 | 11:30 | 94.9 | 12:30 | 96.1 | 11:00 | 94 |
| 12:30 | 88.4 | 12:30 | 95.1 | 13:30 | 95.9 | 11:30 | 94 |
| 13:30 | 88.8 | 13:30 | 95.4 | | | 12:30 | 95 |
| | | | | | | 13:30 | 95 |

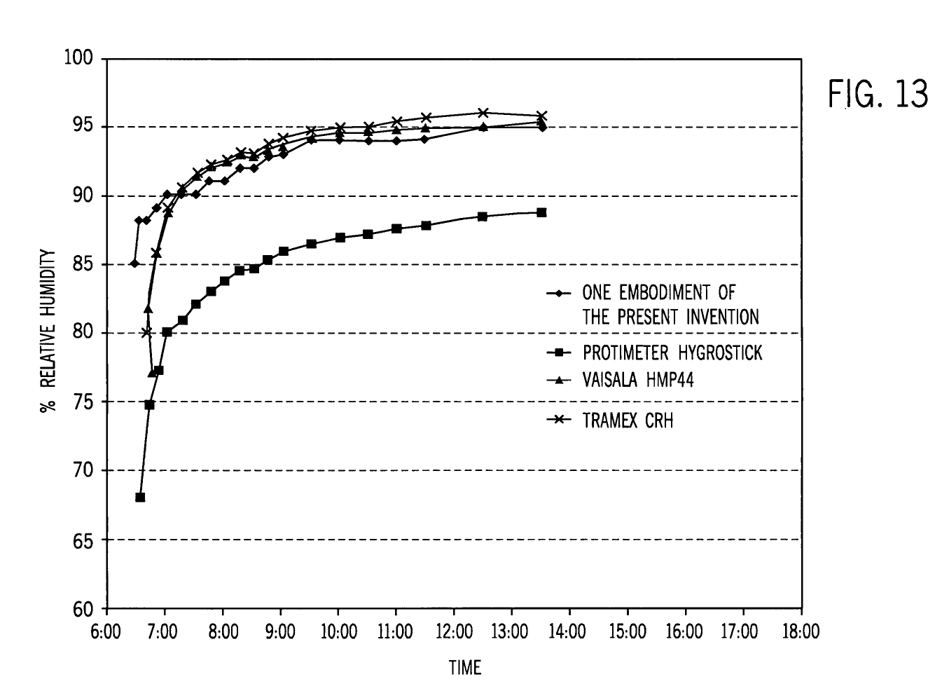

FIG. 13 illustrates equilibration time in a 75% relative humidity sample, Table 3 containing the data used in generating FIG. 13.

TABLE 3
75% RH no vac, 4probes

| | | | Present | |
|---|---|---|---|---|
| | Protimeter | Vaisala | Invention | Tramex |
| TIME | RH % | RH % | RH % | Rh % |
| 9:20 | 69.7 | 76.3 | 87 | 68.2 |
| 9:35 | 75 | 80 | 87 | 75 |
| 9:50 | 77.8 | 80.3 | 85 | 76 |
| 10:05 | 78.7 | 79.7 | 84 | 76.1 |
| 10:20 | 78.7 | 78.9 | 83 | 76.2 |
| 10:35 | 78.7 | 78.4 | 82 | 76.1 |
| 10:50 | 78.5 | 77.8 | 82 | 76 |
| 11:05 | 78.2 | 77.5 | 82 | 75.9 |
| 11:30 | 77.6 | 76.4 | 80 | 75.5 |
| 12:00 | 76.8 | 75.5 | 80 | 75.2 |
| 12:30 | 76.3 | 75.1 | 79 | 75.2 |
| 13:00 | 75.5 | 76.5 | 78 | 75 |
| 13:30 | 75.1 | 73.8 | 78 | 74.7 |
| 14:00 | 74.6 | 73.4 | 77 | 74.3 |
| 14:50 | 74.1 | 72.9 | 77 | 74.1 |

### Example 3

### Relative Humidity Equilibration at Various Vacuum Conditions

A probe in accordance with the principles of the one embodiment of the present invention was utilized to determine the impact of vacuum length on equilibration. A hole was bored in accordance with the principles of the present invention and then, except for the control sample having no vacuum, a vacuum was applied using a standard shopvac-type vacuum with 0.5 to 2.0 inches of water column pressure for the prescribed time in accordance with the principles of the present invention.

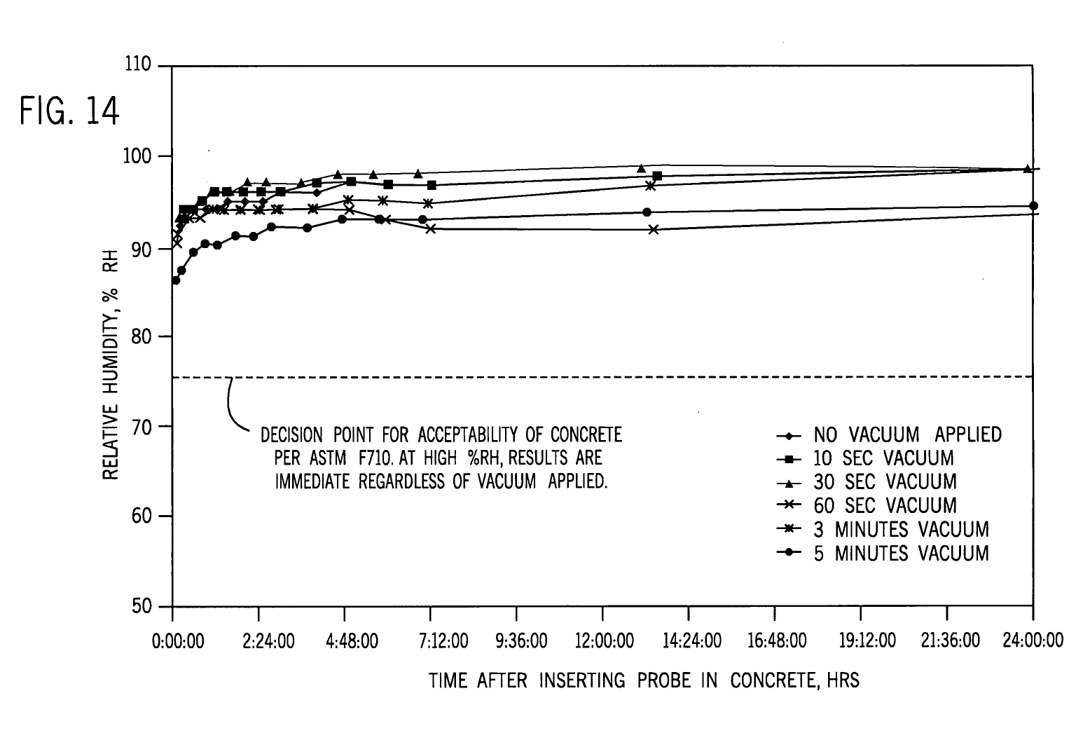

FIG. 14 is a graph of the equilibration times for a probe of one embodiment of the present invention in a sample having high moisture with no vacuum (diamond line), 10 second vacuum (square line), 30 second vacuum (triangle line), 60 second vacuum (x line), 3 minute vacuum (asterisk line), and a 5 minute vacuum (circle line). Table 4 contains the data used in generating FIG. 14.

TABLE 4

| | no vacuum | | | | | | | |
|---|---|---|---|---|---|---|---|---|
| | applied | | 10 sec vacuum | | | 30 sec vacuum | | |
| | Time after | | | Time after | | | Time after | |
| | inserting | | | inserting | | | inserting | |
| TIME | probe, hrs | RH % | TIME | probe, hrs | RH % | TIME | probe, hrs | RH % |
| 5/11/06 9:00 | 0:00:00 | | 5/11/06 9:01 | 0:00:00 | | 5/11/06 9:26 | 0:00:00 | |
| 5/11/06 9:10 | 0:10:00 | 92 | 5/11/06 9:10 | 0:09:00 | 93.0 | 5/11/06 9:30 | 0:04:00 | 91 |
| 5/11/06 9:15 | 0:15:00 | 93 | 5/11/06 9:15 | 0:14:00 | 94.0 | 5/11/06 9:50 | 0:24:00 | 94 |
| 5/11/06 9:20 | 0:20:00 | 93 | 5/11/06 9:20 | 0:19:00 | 94.0 | 5/11/06 10:10 | 0:44:00 | 95 |
| 5/11/06 9:30 | 0:30:00 | 94 | 5/11/06 9:30 | 0:29:00 | 94.0 | 5/11/06 10:30 | 1:04:00 | 96 |
| 5/11/06 9:50 | 0:50:00 | 94 | 5/11/06 9:50 | 0:49:00 | 95.0 | 5/11/06 11:00 | 1:34:00 | 96 |
| 5/11/06 10:10 | 1:10:00 | 94 | 5/11/06 10:10 | 1:09:00 | 96.0 | 5/11/06 11:30 | 2:04:00 | 97 |
| 5/11/06 10:30 | 1:30:00 | 95 | 5/11/06 10:30 | 1:29:00 | 96.0 | 5/11/06 12:00 | 2:34:00 | 97 |
| 5/11/06 11:00 | 2:00:00 | 95 | 5/11/06 11:00 | 1:59:00 | 96.0 | 5/11/06 13:00 | 3:34:00 | 97 |
| 5/11/06 11:30 | 2:30:00 | 95 | 5/11/06 11:30 | 2:29:00 | 96.0 | 5/11/06 14:00 | 4:34:00 | 98 |
| 5/11/06 12:00 | 3:00:00 | 96 | 5/11/06 12:00 | 2:59:00 | 96.0 | 5/11/06 15:00 | 5:34:00 | 98 |
| 5/11/06 13:00 | 4:00:00 | 96 | 5/11/06 13:00 | 3:59:00 | 97.0 | 5/11/06 16:15 | 6:49:00 | 98 |
| 5/11/06 14:00 | 5:00:00 | 97 | 5/11/06 14:00 | 4:59:00 | 97.0 | 5/11/06 22:30 | 13:04:00 | 99 |
| 5/11/06 15:00 | 6:00:00 | 97 | 5/11/06 15:00 | 5:59:00 | 97.0 | 5/12/06 9:15 | 23:49:00 | 99 |
| 5/11/06 16:15 | 7:15:00 | 97 | 5/11/06 16:15 | 7:14:00 | 97.0 | | | |
| 5/11/06 22:30 | 13:30:00 | 98 | 5/11/06 22:30 | 13:29:00 | 98.0 | | | |
| 5/12/06 9:15 | 24:15:00 | 99 | 5/12/06 9:15 | 24:14:00 | 99 | | | |
| 60 sec vacuum | | | 3 minutes vacuum | | | 5 minutes vacuum | | |
| | Time after | | | Time after | | | Time after | |
| | inserting | | | inserting | | | inserting | |
| TIME | probe, hrs | RH % | TIME | probe, hrs | RH % | TIME | probe, hrs | RH % |
| 5/11/06 9:05 | 0:00:00 | | 5/11/06 9:10 | 0:00:00 | | 5/11/06 9:17 | 0:00:00 | |
| 5/11/06 9:10 | 0:05:00 | 90 | 5/11/06 9:15 | 0:05:00 | 91 | 5/11/06 9:20 | 0:03:00 | 86 |
| 5/11/06 9:15 | 0:10:00 | 91 | 5/11/06 9:20 | 0:10:00 | 92 | 5/11/06 9:30 | 0:13:00 | 87 |
| 5/11/06 9:20 | 0:15:00 | 92 | 5/11/06 9:30 | 0:20:00 | 94 | 5/11/06 9:50 | 0:33:00 | 89 |
| 5/11/06 9:30 | 0:25:00 | 93 | 5/11/06 9:50 | 0:40:00 | 94 | 5/11/06 10:10 | 0:53:00 | 90 |
| 5/11/06 9:50 | 0:45:00 | 93 | 5/11/06 10:10 | 1:00:00 | 94 | 5/11/06 10:30 | 1:13:00 | 90 |
| 5/11/06 10:10 | 1:05:00 | 94 | 5/11/06 10:30 | 1:20:00 | 94 | 5/11/06 11:00 | 1:43:00 | 91 |
| 5/11/06 10:30 | 1:25:00 | 94 | 5/11/06 11:00 | 1:50:00 | 94 | 5/11/06 11:30 | 2:13:00 | 91 |
| 5/11/06 11:00 | 1:55:00 | 94 | 5/11/06 11:30 | 2:20:00 | 94 | 5/11/06 12:00 | 2:43:00 | 92 |
| 5/11/06 11:30 | 2:25:00 | 94 | 5/11/06 12:00 | 2:50:00 | 94 | 5/11/06 13:00 | 3:43:00 | 92 |
| 5/11/06 12:00 | 2:55:00 | 94 | 5/11/06 13:00 | 3:50:00 | 94 | 5/11/06 14:00 | 4:43:00 | 93 |
| 5/11/06 13:00 | 3:55:00 | 94 | 5/11/06 14:00 | 4:50:00 | 95 | 5/11/06 15:00 | 5:43:00 | 93 |
| 5/11/06 14:00 | 4:55:00 | 94 | 5/11/06 15:00 | 5:50:00 | 95 | 5/11/06 16:15 | 6:58:00 | 93 |
| 5/11/06 15:00 | 5:55:00 | 93 | 5/11/06 16:15 | 7:05:00 | 95 | 5/11/06 22:30 | 13:13:00 | 94 |
| 5/11/06 16:15 | 7:10:00 | 92 | 5/11/06 22:30 | 13:20:00 | 97 | 5/12/06 9:15 | 23:58:00 | 95 |
| 5/11/06 22:30 | 13:25:00 | 92 | 5/12/06 9:15 | 24:05:00 | 99 | | | |
| 5/12/06 9:15 | 24:10:00 | 94 | | | | | | |

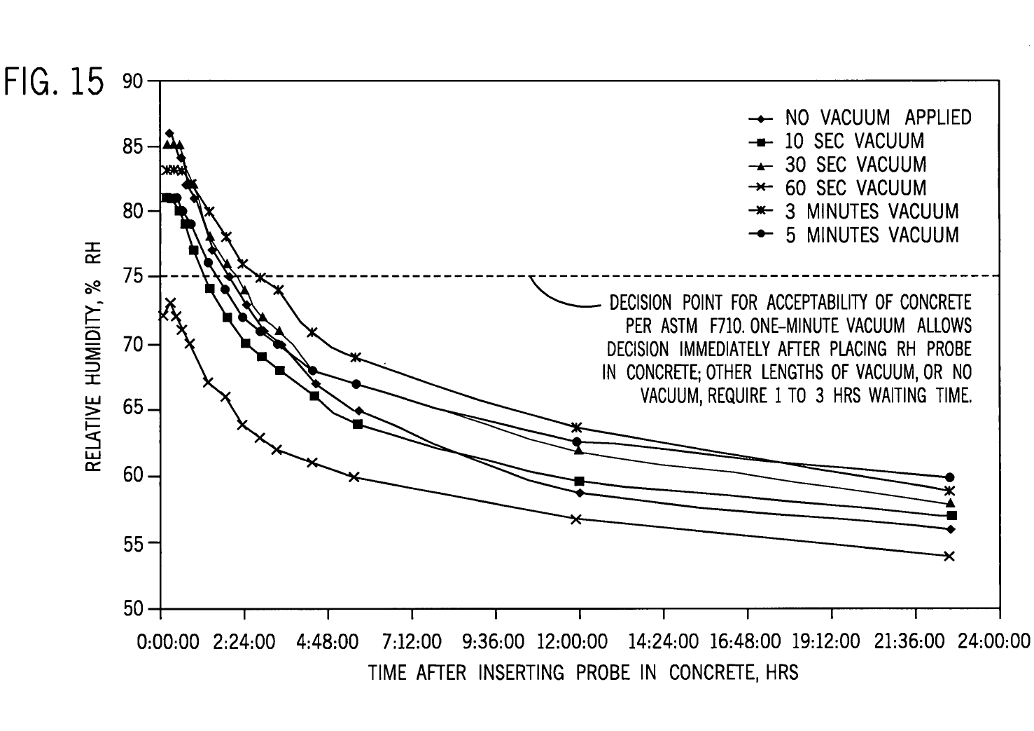

FIG. 15 is a graph of the equilibration times for a probe of one embodiment of the present invention in a sample having low moisture with no vacuum (diamond line), 10 second vacuum (square line), 30 second vacuum (triangle line), 60 second vacuum (x line), 3 minute vacuum (asterisk line), and a 5 minute vacuum (circle line). Table 5 contains the data used in generating FIG. 15.

TABLE 5

| | no vacuum | | 10 sec vacuum | | | | | |
|---|---|---|---|---|---|---|---|---|
| | applied | | | Time | | 30 sec vacuum | | |
| | Time after | | | after | | | Time after | |
| | inserting | | | inserting | | | inserting | |
| TIME | probe, hrs | RH % | TIME | probe, hrs | RH % | TIME | probe, hrs | RH % |
| 5/11/06 10:34 | 0:00:00 | | 5/11/06 10:36 | 0:00:00 | | 5/11/06 10:38 | 0:00:00 | |
| 5/11/06 10:45 | 0:11:00 | 86 | 5/11/06 10:45 | 0:09:00 | 81.0 | 5/11/06 10:45 | 0:07:00 | 85 |
| 5/11/06 10:55 | 0:21:00 | 85 | 5/11/06 10:55 | 0:19:00 | 81.0 | 5/11/06 10:55 | 0:17:00 | 85 |
| 5/11/06 11:05 | 0:31:00 | 84 | 5/11/06 11:05 | 0:29:00 | 80.0 | 5/11/06 11:05 | 0:27:00 | 85 |
| 5/11/06 11:15 | 0:41:00 | 82 | 5/11/06 11:15 | 0:39:00 | 79.0 | 5/11/06 11:15 | 0:37:00 | 83 |
| 5/11/06 11:30 | 0:56:00 | 81 | 5/11/06 11:30 | 0:54:00 | 77.0 | 5/11/06 11:30 | 0:52:00 | 82 |
| 5/11/06 12:00 | 1:26:00 | 77 | 5/11/06 12:00 | 1:24:00 | 74.0 | 5/11/06 12:00 | 1:22:00 | 78 |
| 5/11/06 12:30 | 1:56:00 | 75 | 5/11/06 12:30 | 1:54:00 | 72.0 | 5/11/06 12:30 | 1:52:00 | 76 |
| 5/11/06 13:00 | 2:26:00 | 73 | 5/11/06 13:00 | 2:24:00 | 70.0 | 5/11/06 13:00 | 2:22:00 | 74 |
| 5/11/06 13:30 | 2:56:00 | 71 | 5/11/06 13:30 | 2:54:00 | 69.0 | 5/11/06 13:30 | 2:52:00 | 72 |
| 5/11/06 14:00 | 3:26:00 | 70 | 5/11/06 14:00 | 3:24:00 | 68.0 | 5/11/06 14:00 | 3:22:00 | 71 |
| 5/11/06 15:00 | 4:26:00 | 67 | 5/11/06 15:00 | 4:24:00 | 66.0 | 5/11/06 15:00 | 4:22:00 | 68 |
| 5/11/06 16:15 | 5:41:00 | 65 | 5/11/06 16:15 | 5:39:00 | 64.0 | 5/11/06 16:15 | 5:37:00 | 67 |
| 5/11/06 22:30 | 11:56:00 | 59 | 5/11/06 22:30 | 11:54:00 | 60.0 | 5/11/06 22:30 | 11:52:00 | 62 |
| 5/12/06 9:15 | 22:41:00 | 56 | 5/12/06 9:15 | 22:39:00 | 57.0 | 5/12/06 9:15 | 22:37:00 | 58 |
| | | | | 3 minutes vacuum | | | | |
| | 60 sec vacuum | | | Time | | | 5 minutes vacuum | |
| | Time after | | | after | | | Time after | |
| | inserting | | | inserting | | | inserting | |
| TIME | probe, hrs | RH % | TIME | probe, hrs | RH % | TIME | probe, hrs | RH % |
| 5/11/06 10:40 | 0:00:00 | | 5/11/06 10:45 | 0:05:00 | | 5/11/06 10:51 | 0:11:00 | |
| 5/11/06 10:45 | 0:05:00 | 72 | 5/11/06 10:45 | 0:05:00 | 83 | 5/11/06 10:45 | 0:05:00 | |
| 5/11/06 10:55 | 0:15:00 | 73 | 5/11/06 10:55 | 0:15:00 | 83 | 5/11/06 10:55 | 0:15:00 | 81 |
| 5/11/06 11:05 | 0:25:00 | 72 | 5/11/06 11:05 | 0:25:00 | 83 | 5/11/06 11:05 | 0:25:00 | 81 |
| 5/11/06 11:15 | 0:35:00 | 71 | 5/11/06 11:15 | 0:35:00 | 83 | 5/11/06 11:15 | 0:35:00 | 80 |
| 5/11/06 11:30 | 0:50:00 | 70 | 5/11/06 11:30 | 0:50:00 | 82 | 5/11/06 11:30 | 0:50:00 | 79 |
| 5/11/06 12:00 | 1:20:00 | 67 | 5/11/06 12:00 | 1:20:00 | 80 | 5/11/06 12:00 | 1:20:00 | 76 |
| 5/11/06 12:30 | 1:50:00 | 66 | 5/11/06 12:30 | 1:50:00 | 78 | 5/11/06 12:30 | 1:50:00 | 74 |
| 5/11/06 13:00 | 2:20:00 | 64 | 5/11/06 13:00 | 2:20:00 | 76 | 5/11/06 13:00 | 2:20:00 | 72 |
| 5/11/06 13:30 | 2:50:00 | 63 | 5/11/06 13:30 | 2:50:00 | 75 | 5/11/06 13:30 | 2:50:00 | 71 |
| 5/11/06 14:00 | 3:20:00 | 62 | 5/11/06 14:00 | 3:20:00 | 74 | 5/11/06 14:00 | 3:20:00 | 70 |
| 5/11/06 15:00 | 4:20:00 | 61 | 5/11/06 15:00 | 4:20:00 | 71 | 5/11/06 15:00 | 4:20:00 | 68 |
| 5/11/06 16:15 | 5:35:00 | 60 | 5/11/06 16:15 | 5:35:00 | 69 | 5/11/06 16:15 | 5:35:00 | 67 |
| 5/11/06 22:30 | 11:50:00 | 57 | 5/11/06 22:30 | 11:50:00 | 64 | 5/11/06 22:30 | 11:50:00 | 63 |
| 5/12/06 9:15 | 22:35:00 | 54 | 5/12/06 9:15 | 22:35:00 | 59 | 5/12/06 9:15 | 22:35:00 | 60 |

For low moisture applications, FIG. 15 shows that a 60 second vacuum is optimal in one embodiment.

Given the disclosure of the present invention, one versed in the art would appreciate that there may be other embodiments and modifications within the scope and spirit of the invention. Accordingly, all modifications attainable by one versed in the art from the present disclosure within the scope and spirit of the present invention are to be included as further embodiments of the present invention. The scope of the present invention is to be defined as set forth in the following claims.

What is claimed is:

1. A humidity probe comprising:
a housing having a head portion connected to a tail portion at a first end;
an electronics module disposed within the housing;
a display associated with the head portion and in communication with the electronics module; and
a relative humidity sensor disposed substantially at the second end of the tail portion of the housing and in operative communication with the electronics module and an environment exterior to the humidity probe;
wherein the relative humidity sensor is substantially in direct contact with the exterior environment without the use of dead space within the humidity probe.

2. The humidity sensor of claim 1, wherein the electronics module is disposed within the head portion of the housing.

3. The humidity sensor of claim 2, further comprising a hollow core extending from the electronics module disposed in the head portion through the tail portion and forming an opening at an end of the tail.

4. The humidity sensor of claim 1, further comprising a vapor permeable membrane disposed between the relative humidity sensor and the exterior environment.

5. The humidity sensor of claim 1, wherein the housing is cylindrical in shape.

6. The humidity sensor of claim 1, wherein the circumference of the head portion is substantially larger than the circumference of the tail portion.

7. The humidity sensor of claim 6, further comprising a tapered portion connecting the head portion with the tail portion.

8. The humidity sensor of claim 1, further comprising at least one rib positioned around the perimeter of the housing.

9. A method of monitoring the moisture condition of a building material comprising the steps of:
boring a hole, having a bottom portion and an opening, in a building material;
applying a vacuum to the hole;
inserting into the hole a relative humidity probe having a display and a relative humidity sensor, the relative humidity probe being disposed entirely within the hole;
positioning the probe disposed within the hole with the display located at the top of the hole;
isolating the bottom portion of the hole from an environment outside of the hole to form a test volume;
determining a relative humidity of the substance at the bottom of the hole; and
displaying the relative humidity on the display
wherein the relative humidity sensor is positioned within the probe so as to minimize dead space and is located substantially in contact with the test volume.

10. The method of claim 9, further comprising the step of cleaning the hole with a cleaner attachment.

11. The method of claim 9, wherein the step of boring the hole further comprises providing a lead hole and a counterbore.

12. The method of claim 11, wherein the step of positioning the probe includes countersinking the elongated portion of the probe within the lead hole and the head portion within the counterbore.

13. The method of claim 9, further comprising the step of visually inspecting the display to determine the relative humidity of the substance.

14. The method of claim 9, wherein the vacuum is applied for about 10 second to about 5 minutes.

15. The method of claim 9, wherein the vacuum is applied for about 30 second to about 3 minutes.

16. The method of claim 10, wherein the vacuum is applied for about 60 seconds.

17. The method of claim 9, further comprising the step of providing a remote device in communication with the probe.

18. In a relative humidity probe for insertion into a hole bored in a solid material, the probe comprising a housing having a head portion connected to a tail portion at a first end, an electronics module disposed within the housing, a display associated with the head portion and in communication with the electronics module; and a relative humidity sensor disposed within the tail portion, the improvement comprising
the relative humidity sensor positioned substantially at the second end of the tail portion of the housing to minimize the presence of dead volume;
wherein the relative humidity sensor is substantially in direct contact with the environment of the hole and ribs on the housing seal the hole from environment outside the hole.

19. The humidity sensor of claim 18, wherein the circumference of the head portion is substantially larger than the circumference of the tail portion.

20. The humidity sensor of claim 18, further comprising a transceiver for two-way communication with an exterior communication device.
